(19) United States
(12) Patent Application Publication
(10) Pub. No.: US 2024/0156615 A1
(43) Pub. Date: May 16, 2024

(54) EXPANDABLE FUSION DEVICE FOR POSITIONING BETWEEN ADJACENT VERTEBRAL BODIES

(71) Applicant: Tyler Fusion Technologies, LLC, Tyler, TX (US)

(72) Inventors: Erik Wagner, Austin, TX (US); Michael Schular, Pittsburgh, PA (US)

(21) Appl. No.: 18/372,778

(22) Filed: Sep. 26, 2023

Related U.S. Application Data
(63) Continuation of application No. 17/836,741, filed on Jun. 9, 2022, now Pat. No. 11,766,341.
Application No. 17/836,741 is a continuation of application No. 16/192,932, filed on Nov. 16, 2018, now Pat. No. 11,369,484.
Application No. 16/192,932 is a continuation of application No. 15/351,943, filed on Nov. 15, 2016, now abandoned.
Application No. 15/351,943 is a continuation of application No. 14/185,561, filed on Feb. 20, 2014, now Pat. No. 9,492,288.
(60) Provisional application No. 61/766,982, filed on Feb. 20, 2013.

(51) Int. Cl. A61F 2/44; A61F 2/46

(52) U.S. Cl. CPC A61F 2/447; A61F 2/4455; A61F 2/4611; A61F 2002/2835; A61F 2/4465

(57) ABSTRACT

In some embodiments, system and/or method may include an intervertebral implant for a human spine including an upper body, a lower body, first and second expansion members, and an expansion mechanism. A superior surface of the upper body may function to engage a first vertebra of the human spine. An inferior surface of the lower body may function to engage a second vertebra of the human spine. The first expansion member may include at least a first angled portion positionable, during use, between the upper body and the lower body. The second expansion member may include at least a second angled portion positionable, during use, between the upper body and the lower body. An expansion mechanism may convey, during use, the first and second angled portions in opposing directions increasing a separation distance between the upper body and the lower body.

### PRIORITY CLAIM

This application claims priority to U.S. Provisional Patent Application No. 61/766,982 entitled “EXPANDABLE FUSION DEVICE FOR POSITIONING BETWEEN ADJACENT VERTEBRAL BODIES” filed on Feb. 20, 2013, which is incorporated by reference herein.

### BACKGROUND OF THE INVENTION

### 1. Field of the Invention

This invention relates to spinal implant devices and methods for promoting fusion between adjacent vertebral bodies, and more particularly to expandable fusion devices that can be inserted between adjacent vertebral bodies to facilitate the fusion thereof.

### 2. Description of the Relevant Art

The human spine is a complex mechanical structure, composed of alternating bony vertebrae and fibrocartilaginous discs that are connected by strong ligaments and supported by musculature, that extends from the skull to the pelvis and provides axial support for the body.

The vertebrae generally comprise a vertebral foramen bounded by the anterior vertebral body and the neural arch. The vertebral body comprises two end plates (i.e., superior and inferior) made of thin cartilage overlying a thin layer of hard cortical bone that attaches to the spongy, cancellous interior bone of the vertebral body. The neural arch consists of two pedicles and two lamina that are united posteriorly. The spinous and transverse processes protrude from the neural arch. The superior and inferior articular facets lie at the root of the transverse processes.

The intervertebral discs primarily serve as a mechanical cushion between adjacent vertebral segments of the spinal column and generally comprise two basic components: the annulus fibrosis and the nucleus pulposus. The annulus fibrosis forms the outer perimeter of the disc and is a tough ring that binds adjacent vertebrae together. The nucleus pulposus fills the interior of the disc and carries load.

The spine as a whole is a highly flexible structure capable of a high degree of curvature and twist in nearly every direction. However, genetic or developmental irregularities, trauma, chronic stress, and degenerative wear can result in spinal pathologies for which surgical intervention may be necessary.

It is common practice to remove a spinal disc in cases of spinal disc deterioration, disease or spinal injury. More particularly, the discs sometimes become diseased or damaged such that the height of the disc is reduced, which causes the annulus to buckle in areas where the laminated plies are loosely bonded. As the overlapping laminated plies of the annulus begin to buckle and separate, circumferential and/or radial annular tears may occur, allowing nucleus material to escape or form a bulge in the annulus. Such disruption to the natural intervertebral separation and the resulting herniation produces pain, which can be alleviated by removal of the disc and restoration of the natural separation distance. In cases of chronic back or leg pain resulting from a degenerated or herniated disc, removal of the disc can become the desired course of treatment.

In some cases it is desired to fuse the adjacent vertebrae together after removal of the disc. Such a procedure is sometimes referred to as “intervertebral fusion” or “interbody fusion”.

Many techniques and instruments have been devised to perform intervertebral fusion. There is common agreement that the strongest intervertebral fusion is interbody fusion between the lumbar bodies, which may be augmented by a posterior or facet fusion. In cases of intervertebral fusion, either structural bone, or a rigid interbody fusion “cage” typically filled with morselized bone, is placed centrally within the space where the spinal disc once resided. Multiple bony grafts or cages may be used within that space. Furthermore, multiple surgical approaches may be utilized, including anterior, posterior, or lateral surgical approaches.

Such practices are characterized by certain disadvantages, including the need to distract the disc space in order to implant the fusion device and thereby restore the diseased disc space to its normal or healthy height. However, it can be difficult to distract the adjacent vertebral bodies sufficiently to easily insert the fusion device between adjacent vertebral bodies. As a result, it is often necessary to drive the fusion device into the space between the vertebral bodies using impaction with a mallet and the application of significant force. The use of such impaction and force increases the risk of damage to local soft tissue such as blood vessels and the surrounding nerves, and can lead to suboptimal placement and/or failure of the insertion instrumentation. Furthermore, the use of such impaction and force can damage or compromise the vertebral endplates, resulting in eventual failure and subsidence of the fusion device into the vertebral bodies and hence loss of disc height.

Therefore, there is a need for a fusion device that can be placed between adjacent vertebral bodies at minimal height and, thereafter, be variably adjusted with minimal force application to the preferred height for an individual patient. Furthermore, it is desirable that the expandable fusion device be maintained in a closed (i.e., unexpanded) position during insertion and handling, and that it be rigidly attachable to a holder so as to facilitate maximum control by the surgeon during insertion and deployment.

### SUMMARY

Accordingly, there is now provided an expandable fusion device that can be placed between adjacent vertebral bodies at minimal height and, thereafter, be variably adjusted with minimal force application to the preferred height for an individual patient. In one embodiment, an expandable PLIF (Posterior Lumbar Interbody Fusion) device or an expandable TLIF (Transforaminal Lumbar Interbody Fusion) device, is disclosed. The expandable fusion device generally includes: a cage, superior and lower bodys, and an expansion mechanism with opposing proximal and second expansion members. The application of torque to the expansion mechanism in one direction causes the proximal and second expansion members to separate, whereby to move the superior and lower bodies away from one another and hence increase the height of expandable fusion device 5. The application of torque to the expansion mechanism in the opposite direction causes the proximal and second expansion members to approach one another, whereby to move the superior and lower bodys toward one another and hence decrease the height of the expandable fusion device.

Further embodiments may include: (i) angled or lordotic superior and lower bodys to match the angle of the disc space; (ii) mismatched proximal and second expansion members, such that the anterior portion of the expandable fusion device opens more than the posterior portion of the expandable fusion device, thereby resulting in a fusion device that increases in both height and lordosis; (iii) dual or multiple expansion mechanisms for anterior spinal approaches; (iv) a curved or flexible holder for the expandable fusion device for oblique access approaches; and (v) additional angled components (i.e., intermediate the aforementioned proximal and second expansion members) for longer expandable fusion devices.

In some embodiments, system and/or method may include an intervertebral implant for a human spine including an upper body, a lower body, first and second expansion members, and an expansion mechanism. The upper body may include an inferior surface and a superior surface. The superior surface of the upper body may function to engage a first vertebra of the human spine. The lower body may include a superior surface and an inferior surface. The inferior surface of the lower body may function to engage a second vertebra of the human spine. The first expansion member may include at least a first angled portion. The first angled portion may be positionable, during use, between the inferior surface of the upper body and the superior surface of the lower body. At least the first angled portion may be oriented towards a first end of the intervertebral implant. The second expansion member may include at least a second angled portion positionable, during use, between the inferior surface of the upper body and the superior surface of the lower body. At least the second angled portion may be oriented towards a second end of the intervertebral implant. At least the second angled portion may be oriented in an opposing direction relative to at least the first angled portion. An expansion mechanism may convey, during use, the first and second angled portions in opposing directions increasing a separation distance between the upper body and the lower body. The first and/or second angled portion may include a wedge-shaped portion.

In some embodiments, the expansion mechanism may include a threaded elongated member. The threaded elongated member may include a proximally threaded portion. The first expansion member may include a threaded opening which the threaded portion of the elongated member engages, during use.

In some embodiments, a distal end of the elongated member engages, during use, a proximal end of the second expansion member. The distal end may engage a recess in the second expansion member and rotates freely within it.

In some embodiments, the expansion mechanism may include a first elongated member and a second elongated member. The first elongated member may include a proximally threaded portion. The first expansion member may include a threaded opening which the threaded portion of the first elongated member engages, during use. The second elongated member may be positionable, during use, in an opening in the second expansion member. A distal end of the first elongated member may engage, during use, a proximal end of the second elongated member. In some embodiments, a distal end of the first elongated member may engage, during use, a proximal end of the second elongated member such that the distal end of the first elongated member is positioned in the opening in the second expansion member.

In some embodiments, the expansion member may include a locking member. The locking member may be positionable in the second expansion member such that the distal end of the first elongated member is inhibited, during use, from removal from the opening in the second expansion member.

In some embodiments, the intervertebral implant may include a cage. The cage may form a perimeter around the intervertebral implant in which at least portions of the upper body, the lower body, the first expansion member, the second expansion member, and the expansion mechanism are positioned, during use, in the cage. The cage may include one or more openings along the perimeter to allow graft material to be positioned during use.

In some embodiments, a lateral cross section of a perimeter of the intervertebral implant may include a curved shape such that at least a first portion of the perimeter is substantially convex and at least a second portion of the perimeter is substantially concave, wherein the second portion is substantially opposite the first portion.

In some embodiments, the upper body and/or the lower body may include an opening wherein graft material is positionable during use. The upper body and/or the lower body may include an opening which increases in size as the first and second angled portions are conveyed in opposing directions.

In some embodiments, the superior surface of the upper body and/or the inferior surface of the lower body may include protrusions (e.g., teeth). The protrusions may promote, during use, retention of the implant between the first vertebra and the second vertebra after insertion.

### BRIEF DESCRIPTION OF THE DRAWINGS

Advantages of the present invention may become apparent to those skilled in the art with the benefit of the following detailed description of the preferred embodiments and upon reference to the accompanying drawings.

FIG. 1 depicts a schematic side view showing an expandable fusion device formed in accordance with the present invention, with the expandable fusion device being disposed between adjacent vertebral bodies.

FIG. 2 depicts a schematic exploded view of an expandable fusion device.

FIG. 3 depicts a schematic front perspective view of an expandable fusion device, with the expandable fusion device being shown in an unexpanded position.

FIG. 4 depicts a schematic front perspective view of an expandable fusion device, with the expandable fusion device being shown in an expanded position.

FIG. 5 depicts a schematic rear perspective view of an expandable fusion device, with the expandable fusion device being shown in an unexpanded position.

FIG. 6 depicts a schematic rear perspective view of an expandable fusion device, with the expandable fusion device being shown in an expanded position.

FIG. 7 is a schematic side view of an expandable fusion device, with the expandable fusion device being shown in an unexpanded position.

FIG. 8 depicts a schematic side view of an expandable fusion device, with the expandable fusion device being shown in an expanded position.

FIG. 9 depicts a schematic top view of an expandable fusion device.

FIG. 10 depicts a schematic side cross-sectional view of an expandable fusion device, with the expandable fusion device being shown in an unexpanded position.

FIG. 11 depicts a schematic side cross-sectional view of an expandable fusion device, with the expandable fusion device being shown in an expanded position.

FIG. 12 depicts a schematic perspective cross-sectional view of an expandable fusion device, with the expandable fusion device being shown in an unexpanded condition.

FIG. 13 depicts a schematic perspective view of a lower body.

FIG. 14 depicts a schematic perspective cross-sectional view of a lower body.

FIG. 15 depicts a schematic perspective cross-sectional view of an upper body.

FIG. 16 depicts a schematic right side perspective view of the proximal and second expansion members.

FIG. 17 depicts a schematic left side perspective view of the proximal and second expansion members.

FIG. 18 depicts a schematic view showing insertion instruments for use with an expandable fusion device.

FIG. 19 depicts a schematic transparent side view of an expandable fusion device.

FIG. 20 depicts a schematic exploded view of an expandable fusion device.

FIG. 21 depicts a schematic cross-sectional view of a curved expandable fusion device in an unexpanded state.

FIG. 22 depicts a schematic cross-sectional view of a curved expandable fusion device in an expanded state.

FIG. 23 depicts a schematic view of a curved expandable fusion device as the device is being inserted between two adjacent vertebrae.

FIG. 24 depicts a schematic view showing insertion instruments for use with an expandable fusion device.

FIG. 25 depicts a schematic view showing a distal end of an insertion instrument for use with an expandable fusion device.

FIG. 26 depicts a schematic view showing a proximal end of an insertion instrument for use with an expandable fusion device.

FIG. 27 depicts a schematic view of a disposable expandable fusion implant insertion device.

FIG. 28 depicts a schematic transparent view of a disposable expandable fusion implant insertion device.

FIG. 29 depicts a schematic perspective view of an expandable fusion implant in an expanded state wherein an upper body portion of the implant is depicted as transparent.

FIG. 30 depicts a schematic perspective view of an expandable fusion implant in an expanded state wherein an upper body portion of the implant is depicted as transparent.

FIG. 31 depicts a schematic perspective view of an expandable fusion implant in a contracted state.

FIG. 32 depicts a schematic perspective view of an expandable fusion implant in a contracted state coupled to an insertion instrument with portions of the insertion instruments depicted as transparent. An upper body of the implant is not depicted and a second expandable member is depicted as transparent.

FIG. 33 depicts a schematic view of an insertion instrument with an expandable fusion device.

FIG. 34 depicts a schematic view of a distal end of an insertion instrument with an expandable fusion device.

FIG. 35 depicts a schematic view of a distal end of an insertion instrument with an expandable fusion device with a portion of the insertion instrument removed for clarity.

FIG. 36 depicts a schematic view of a distal end of an insertion instrument with an expandable fusion device with a portion of the insertion instrument removed for clarity.

FIG. 37 depicts a schematic perspective view of an expandable fusion implant in a contracted state. At least an upper body and a cage of the implant is not depicted.

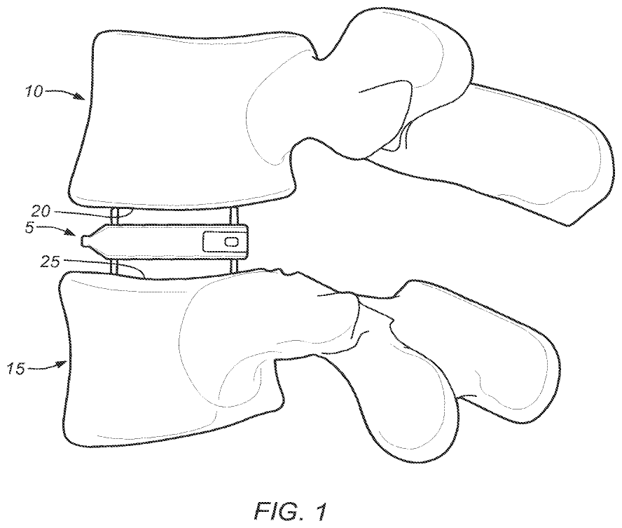

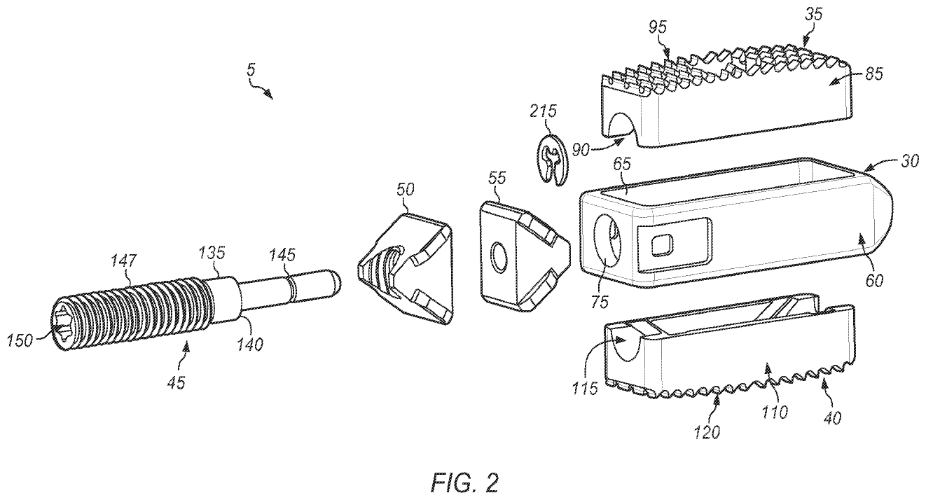

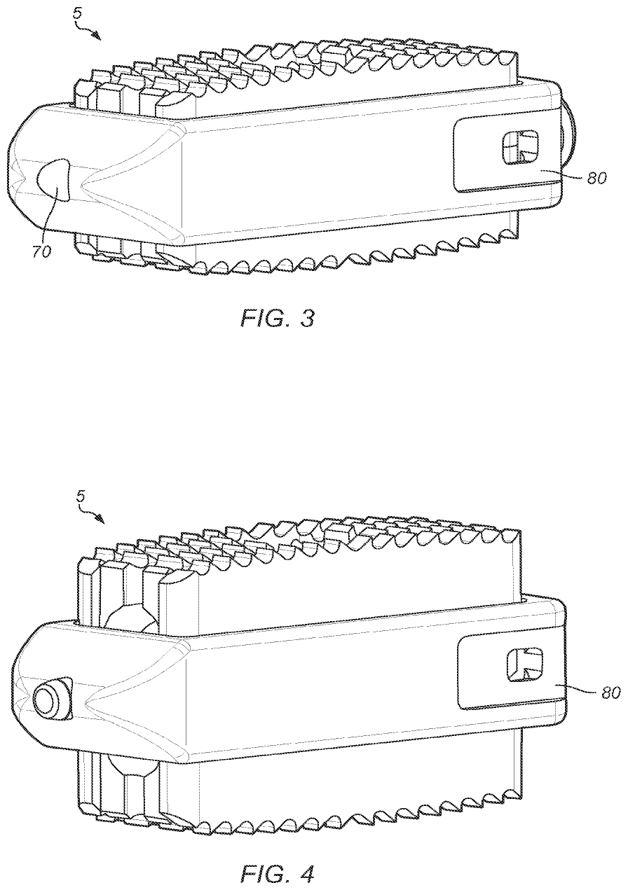

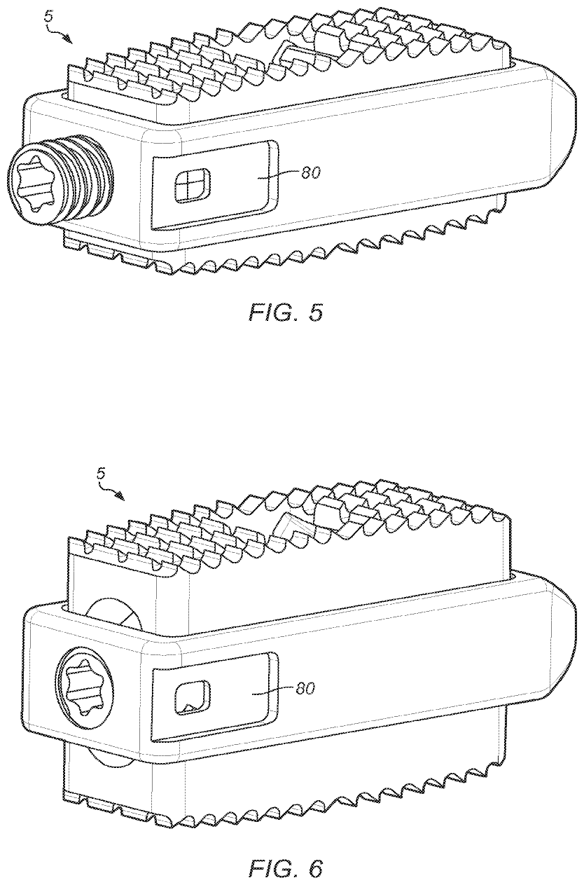

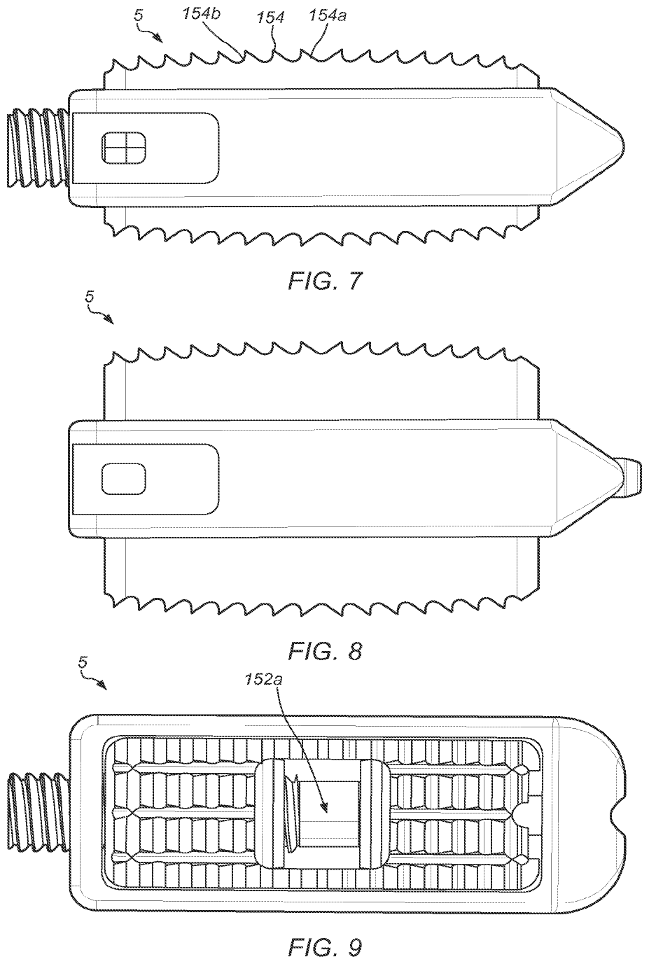

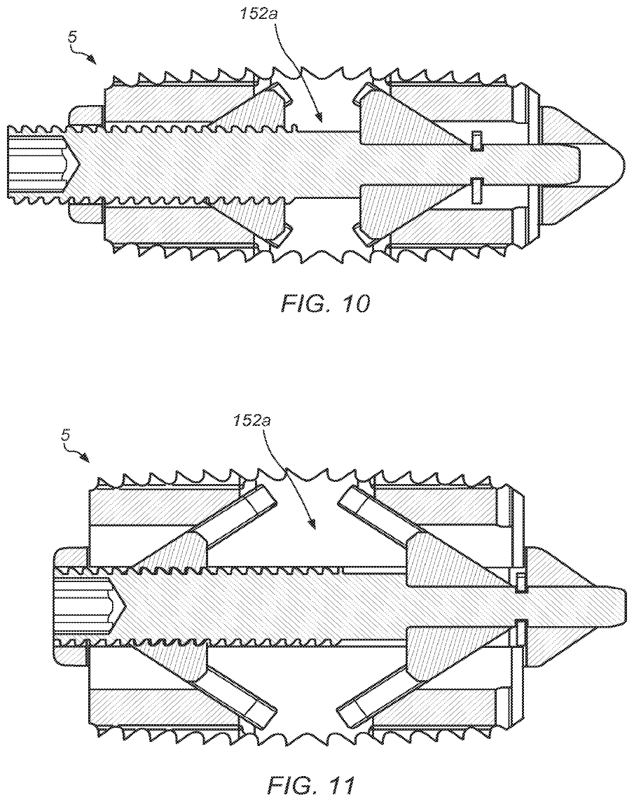

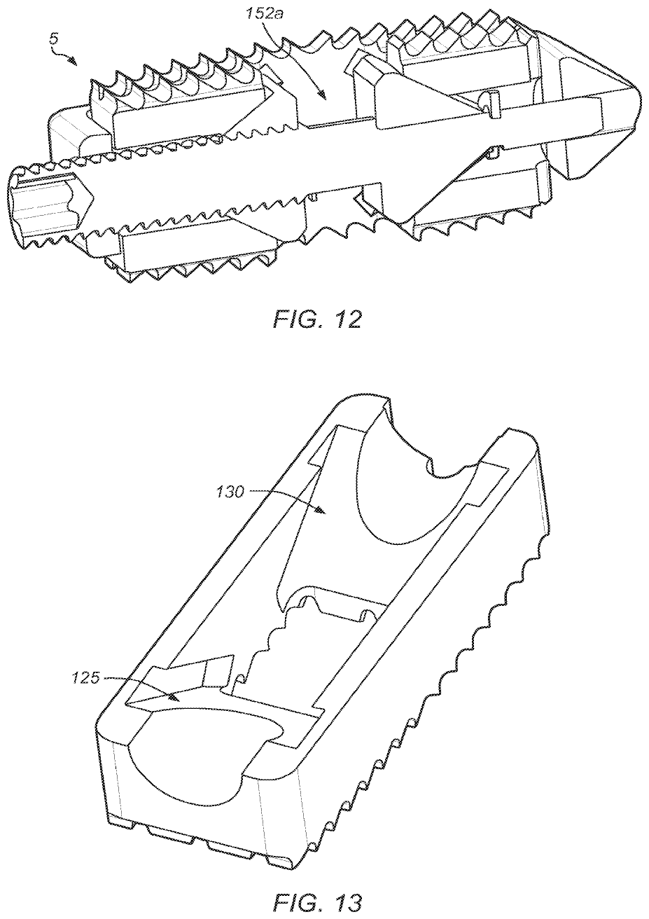

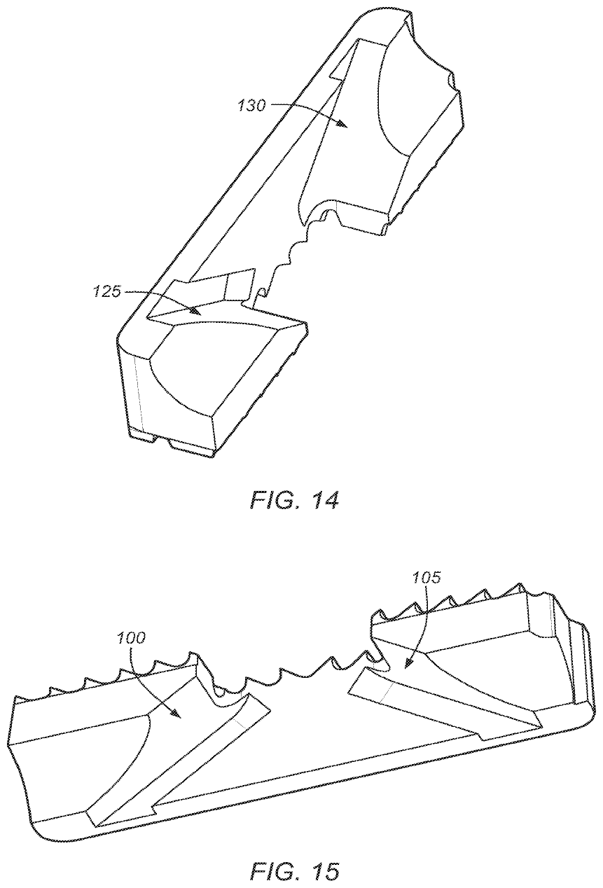

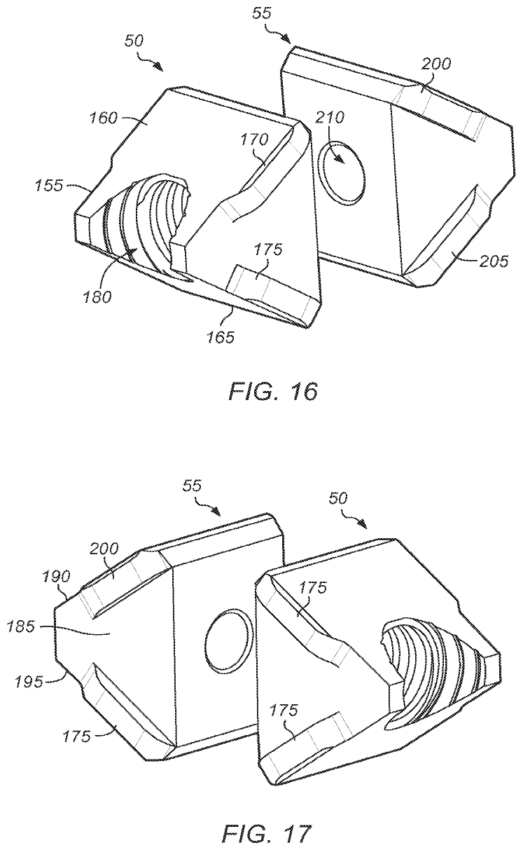

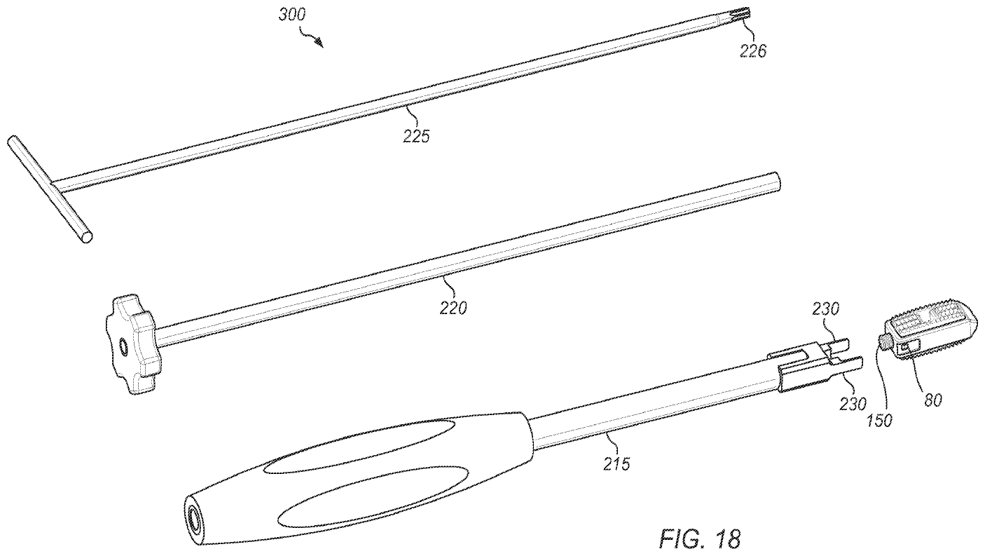

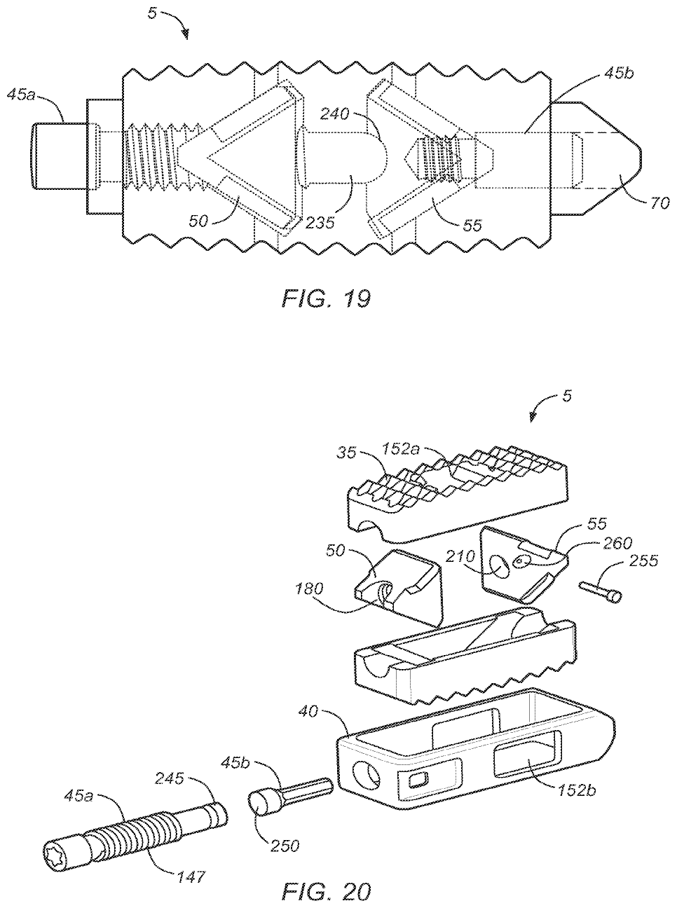

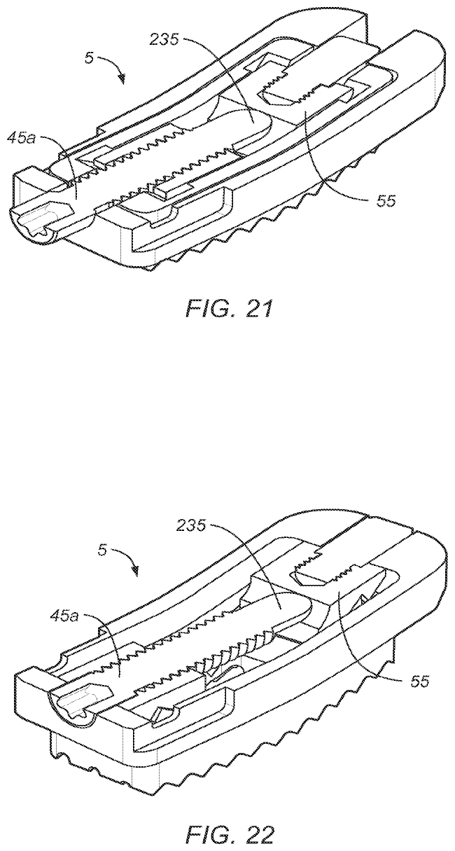

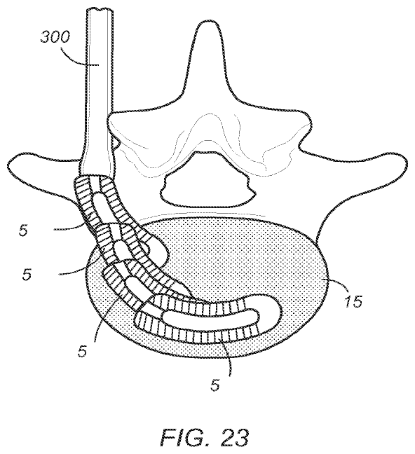

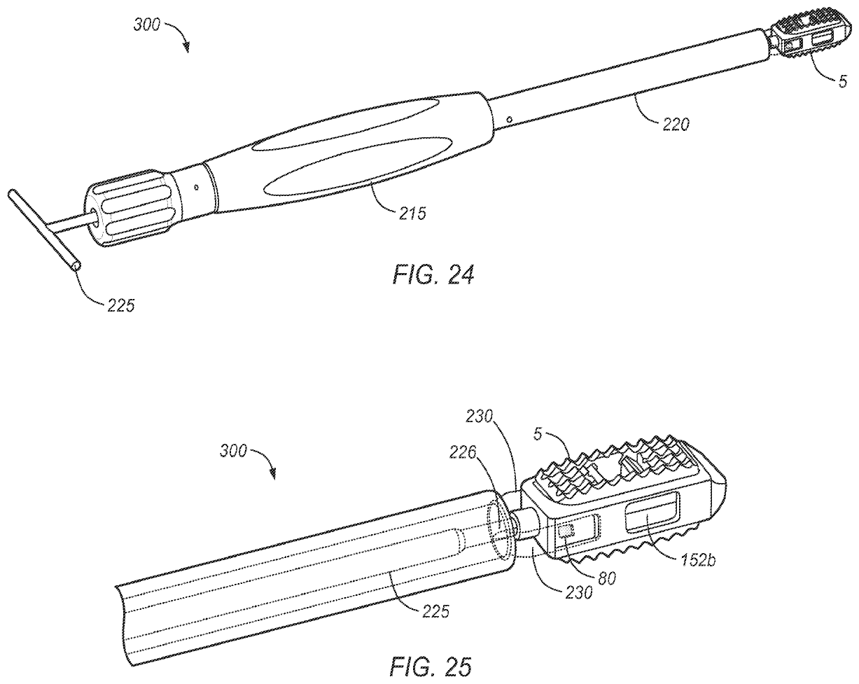

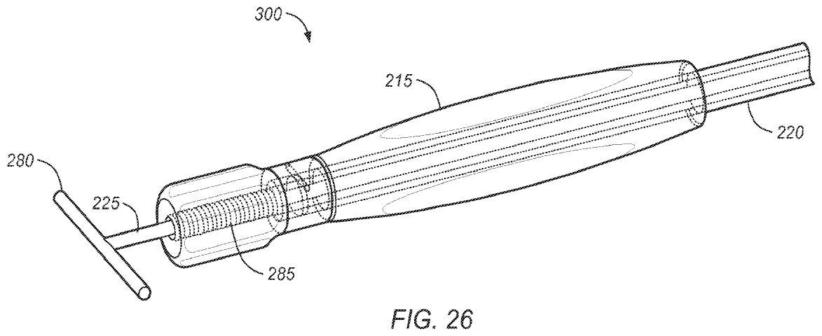

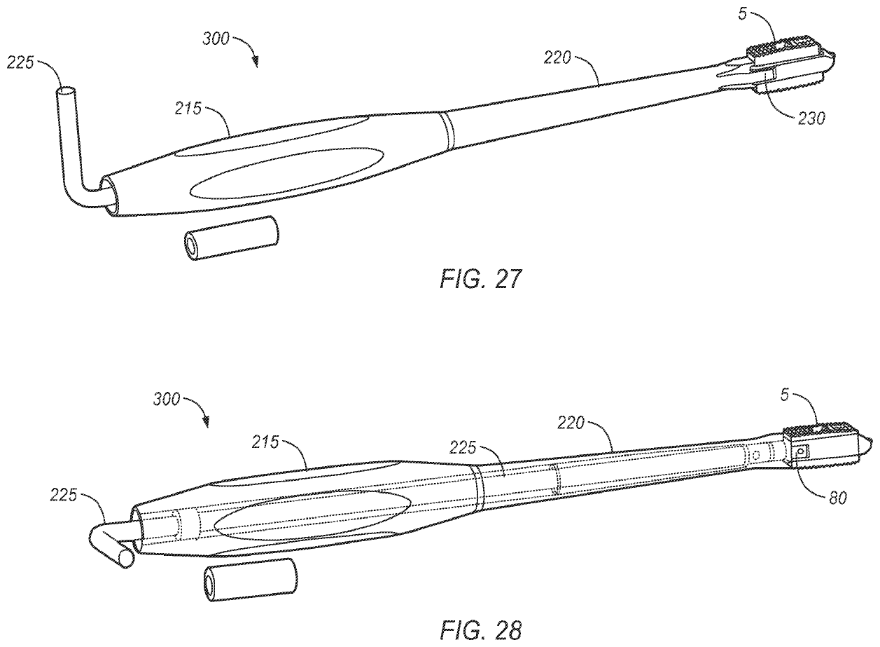

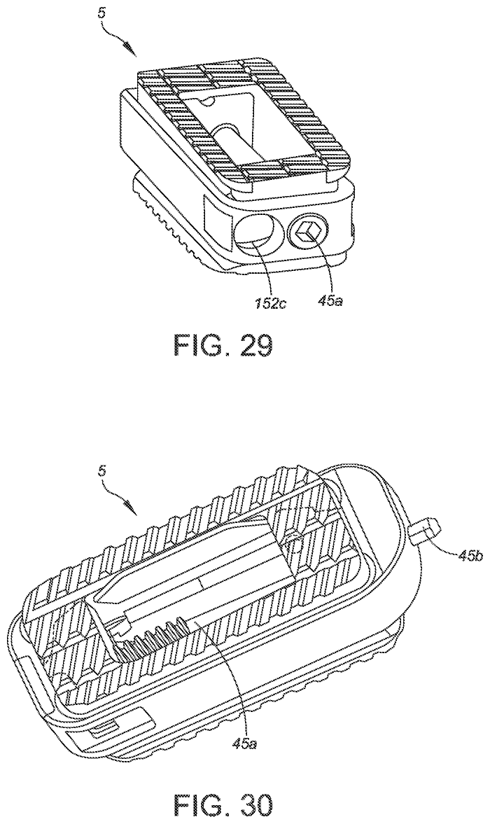

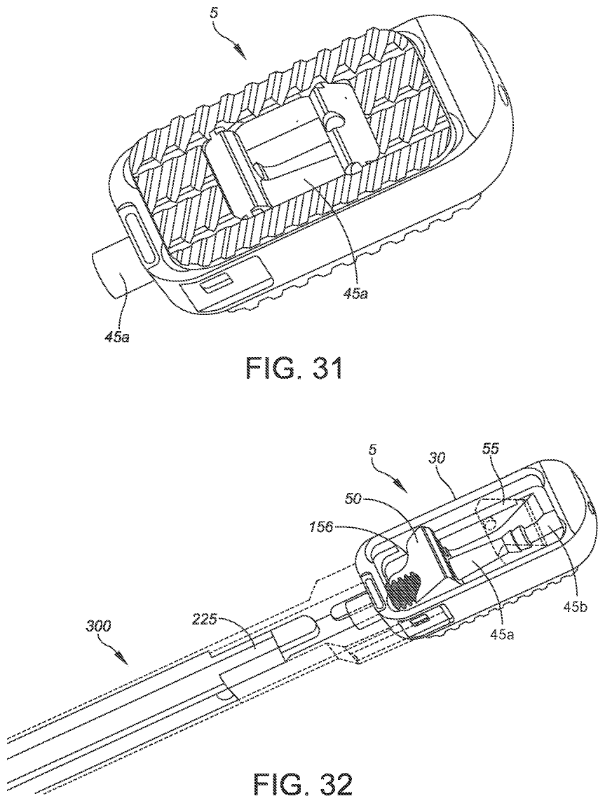

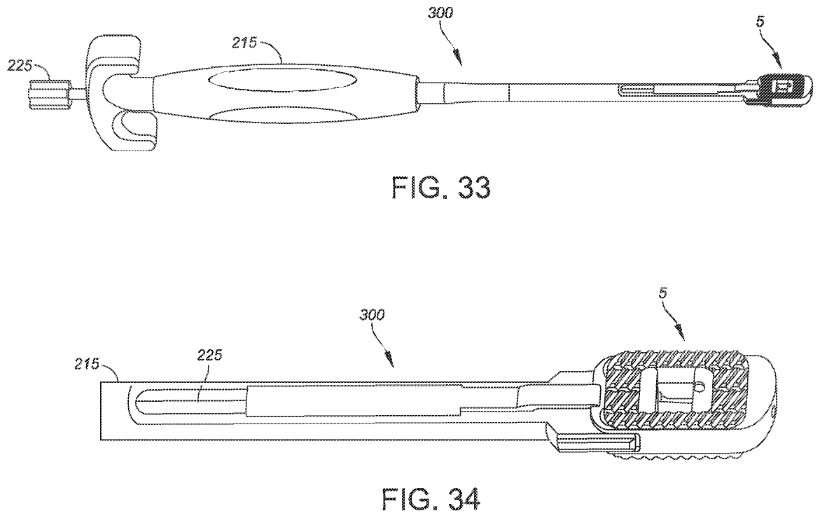

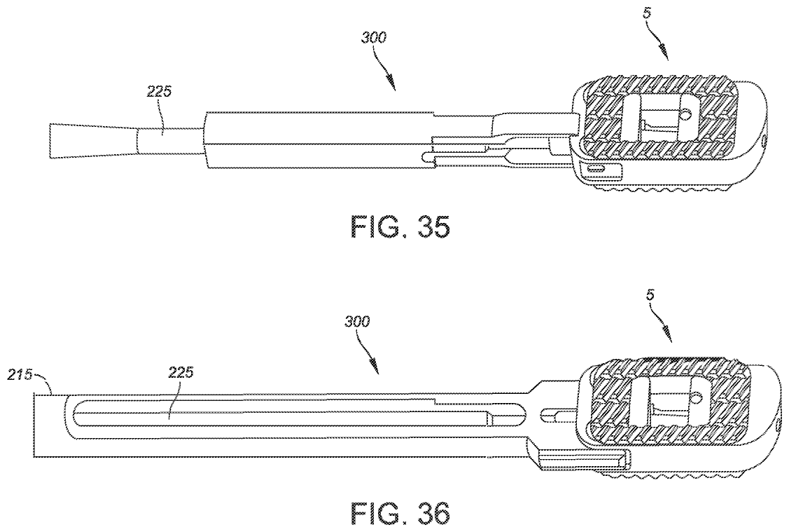

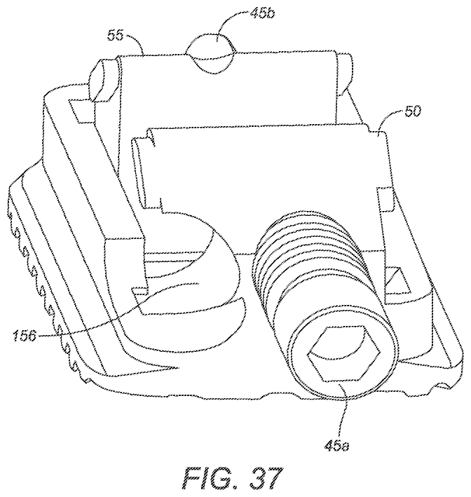

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third die electrically connected to the module substrate” does not preclude scenarios in which a “fourth die electrically connected to the module substrate” is connected prior to the third die, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112, paragraph six, interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an and “the” include singular and plural referents unless the content clearly dictates otherwise. Thus, for example, reference to “a linker” includes one or more linkers.

1-20. (canceled)

21. An intervertebral implant comprising:
a first wedge;
a second wedge;
an upper body engaged with the first wedge and the second wedge;
a lower body engaged with the first wedge and the second wedge;
a cage housing the first and second wedges, wherein the cage comprises a first proximal opening and a second proximal opening; and
a drive gear operatively engaged with the first wedge via the first proximal opening to move the first and second wedges in opposite directions.

22. The intervertebral implant of claim 21, wherein at least one of the first and second wedges is positioned off center.

23. The intervertebral implant of claim 22, wherein the first wedge comprises an opening.

24. The intervertebral implant of claim 22, wherein the drive gear is positioned off center.

25. The intervertebral implant of claim 21, wherein the drive gear comprises:
a worm gear; and
a shaft extending from the worm gear.

26. The intervertebral implant of claim 25, wherein the drive gear further comprises a stop engaged with the first wedge.

27. The intervertebral implant of claim 21, wherein the implant is filled with biological material.

28. The intervertebral implant of claim 21, wherein the upper body includes at least one pair of recessed tracks.

29. The intervertebral implant of claim 21, wherein the lower body includes at least one pair of recessed tracks.

30. The intervertebral implant of claim 21, wherein the intervertebral implant comprises a curved cross-section.

31. An intervertebral implant system comprising:
an intervertebral implant comprising:
a first wedge;
a second wedge;
an upper body engaged with the first wedge and the second wedge;
a lower body engaged with the first wedge and the second wedge;
a cage housing the first and second wedges, wherein the cage comprises a first proximal opening and a second proximal opening; and
a drive gear operatively engaged with the first wedge via the first proximal opening to move the first and second wedges in opposite directions; and
an implant insertion device.

32. The intervertebral implant system of claim 31, wherein the cage further comprises a first seat on a first side of the cage and a second seat on a second, opposing side of the cage.

33. The intervertebral implant system of claim 32, wherein the implant insertion device comprises:
a handle comprising:
a first extension and a second extension for positioning in the first and second seats of the cage;
at least one holder for releasably securing the intervertebral implant to the handle; and
an engaging member for operatively engaging the drive gear of the intervertebral implant.

34. The intervertebral implant system of claim 31, wherein the implant insertion device is disposable.

35. The intervertebral implant system of claim 33, wherein the at least one holder is spring loaded such that the at least one holder is biased to apply pressure to a surface of the intervertebral implant during use.

36. A method for manipulating the spacing between two adjacent vertebral bodies, the method comprising:
removing at least a portion of a disc between two vertebrae of a spine to create a space between the two vertebrae;
positioning an intervertebral implant in the space between the two vertebrae, wherein the intervertebral implant comprises:
a first wedge;
a second wedge;
an upper body engaged with the first wedge and the second wedge;
a lower body engaged with the first wedge and the second wedge; and
a drive gear operatively engaged with the first wedge to move the first and second wedges in opposite directions;
rotating the drive gear in a first direction so as to move the first and second wedges in opposite directions so as to increase a separation distance between the upper body and the lower body.

37. The method of claim 36, wherein the drive gear further comprises a stop engaged with the first wedge.

38. The method according to claim 37 further comprising rotating the drive gear in a second direction so as to move the first and second wedges in the same direction so as to decrease the separation distance between the upper body and the lower body.

39. The method of claim 36, wherein the drive gear comprises:
a worm gear; and
a shaft extending from the worm gear.

40. The method of claim 36, wherein the intervertebral implant further comprises a cage housing the first and second wedges, wherein the cage comprises a first proximal opening for receiving the drive gear and a second proximal opening.
